(19) United States
(12) Patent Application Publication
(10) Pub. No.: US 2014/0207047 A1
(43) Pub. Date: Jul. 24, 2014

(54) TRANSDERMAL DRUG DELIVERY SYSTEM AND METHOD

(71) Applicant: Chrono Therapeutics, Inc., Cambridge, MA (US)

(72) Inventors: Guy DiPierro, Cambridge, MA (US); Michael Barsness, Oxford, MA (US); Bireswar Chakroborty, Andover, MA (US); Kuowei Chang, Lexington, MA (US)

(73) Assignee: Chrono Therapeutics, Inc., Cambridge, MA (US)

(21) Appl. No.: 14/156,944

(22) Filed: Jan. 16, 2014

Related U.S. Application Data
(60) Provisional application No. 61/755,307, filed on Jan. 22, 2013.

(51) Int. Cl. A61M 35/00

(52) U.S. Cl. CPC A61M 35/00
USPC 604/20; 604/304; 604/173; 604/22; 604/113; 604/290; 604/68; 604/506; 604/500

(57) ABSTRACT

A bioactive agent delivery device having a plurality of reservoirs wherein each reservoir houses a solvent provides a means for separable and segregated delivery of bioactive agent(s) to a patient. The device can include a plurality of absorbent materials wherein each absorbent material is pretreated with a bioactive agent and a delivery mechanism operable to deliver to any of the plurality of absorbent materials a controlled portion of solvent from any of a plurality of reservoirs. A diffusion layer interposed between the absorbent materials and an epidermis transfers the bioactive agent from the absorbent materials to the epidermis for delivery of the bioactive agent.

### RELATED APPLICATION

The present application relates to and claims the benefit of priority to U.S. Provisional Patent Application No. 61/755,307 filed Jan. 22, 2013 which is hereby incorporated by reference in its entirety for all purposes as if fully set forth herein.

### BACKGROUND OF THE INVENTION

1. Field of the Invention

Embodiments of the present invention relate, in general, to controlled delivery of biologically active agents and, more particularly, to methodology and systems for bio-synchronous transdermal drug delivery.

2. Relevant Background

In the field of drug delivery, it is recognized that supplying a bioactive agent in an appropriate temporal pattern maximizes efficacy and minimizes toxicity. Controlled release drug delivery systems are intended to improve response to a drug and/or lessen side effects of a drug. The term “controlled release” refers generally to delivery mechanisms that make an active ingredient available to the biological system of a host in a manner that supplies the drug according to a desired temporal pattern. Controlled release drug delivery may be implemented using immediate release systems, delayed release systems, and sustained release systems. In most cases, controlled release systems are designed to maintain a sustained plasma level of a bioactive agent within a human or animal host over a period of time.

Immediate release refers to systems that make the active ingredient available immediately after administration to the bio-system of the host. Immediate release systems include continuous or pulsed intravenous infusion or injections. Such systems provide a great deal of control because administration can be both immediately started and stopped and the delivery rate can be controlled with great precision. However, the administration is undesirably invasive as they generally involve administration via a puncture needle or catheter.

Delayed release refers to systems in which the active ingredient is made available to the host at some time after administration. Such systems include oral as well as injectable drugs in which the active ingredient is coated or encapsulated with a substance that dissolves at a known rate so as to release the active ingredient after the delay. Unfortunately, it is often difficult to control the degradation of the coating or encapsulate after administration and actual performance will vary from patient to patient.

Sustained release generally refers to release of an active ingredient such that the level of active ingredient available to the host is maintained at some level over a period of time. Like delayed release systems, sustained release systems are difficult to control and exhibit variability from patient to patient. Due to absorption through the gastrointestinal tract, drug concentrations rise quickly in the body when taking a pill, but the decrease is dependent on excretion and metabolism, which cannot be controlled. In addition, the absorption through the gastrointestinal tract may lead to considerable side effects (such as ulcers) and may severely damage the liver.

Transdermal drug delivery has developed primarily for sustained release of drugs in situations where oral sustained release systems are inadequate. In some cases, drugs cannot be effectively administered orally because the active ingredients are destroyed or altered by the gastrointestinal system. In other cases the drug may be physically or chemically incompatible with the coatings and/or chelating agents used to implement sustained release. In other cases, transdermal delivery systems may provide sustained release over a period of days or weeks whereas orally administered drugs may offer sustained performance over only a few hours. A wide variety of active substances can be delivered through transdermal systems provided the active substance can permeate the skin barrier.

In most cases transdermal delivery systems are passive, taking the form of a patch that is adhesively attached to the host. The patch includes a quantity of the active substance, along with a suitable carrier if need be, absorbed in a sponge or similar system. Once applied, the active ingredient diffuses into the host through the skin at a rate determined by the concentration of the active substance and the diffusivity of the active substance. However, a variety of physical and chemical processes at the skin/patch boundary affect the delivery rate and may eventually inhibit drug delivery altogether.

Active transdermal delivery systems have been developed to help regulate the delivery rate by providing mechanisms to improve drug delivery over time by “pumping” the active ingredient. One such system is described in U.S. Pat. No. 5,370,635 entitled “DEVICE FOR DELIVERING A MEDICAMENT” which describes a system for delivering a medicament and dispensing it to an organism for a relatively long period of time, for example at least a few days. This device can be adapted for positioning on the surface of the skin of a human or an animal in order to apply a medicament thereto from the outer side thereof.

Conventional transdermal systems circumvent the disadvantages of absorption through the gastrointestinal tract, but they do not optimize or tailor the dosing regimen to offset peak symptoms. In addition the constant transdermal delivery of a drug can lead to severe side effects, including debilitating sleep disorders and ever increasing tolerance.

Timed gastrointestinal delivery is most often used to maintain a sustained level of a drug in the body. A significant focus of current research in bioactive agent delivery has been to determine the influence of a patient's circadian or other biological rhythms on drug efficacy and efficiency. This research demonstrates that certain disease symptoms follow a daily pattern, with peak symptoms at particular times of the day. It has been widely acknowledged that hormones, neurotransmitters and other intra-body compounds are released in varying amounts at different times of the day pursuant to daily patterns. The Wall Street Journal reported on May 27, 2003 that ‘doctors are increasingly looking at the clock when it comes to prescribing medicine, instructing patients not only regarding what drug to use but also precisely when to take it’. The new approach stems from a growing body of research demonstrating that distinct disease symptoms tend to get worsen at specific times of the day. By synchronizing medications with a patient's body clock, many physicians believe that the drugs will work more effectively and with fewer side effects. In some cases, the improvements have been so pronounced that doctors have been able to reduce dosages.

American Pharmacy reports that ‘circadian physiologic processes alter drug absorption, distribution, metabolism, and excretion.’ The onset and symptoms of diseases such as asthma attacks, coronary infarction, angina pectoris, stroke and ventricular tachycardia are circadian phase dependent. In humans, variations during the 24 hour day in pharmacokinetics (chronopharmacokinetics) have been shown for cardiovascular active drugs (propranolol, nifedipine, verapamil, enalapril, isosorbide 5-mononitrate and digoxin), anti-asthmatics (theophylline and terbutaline), anticancer drugs, psychotropics, analgesics, local anesthetics and antibiotics, to mention but a few. Even more drugs have been shown to display significant variations in their effects throughout the day (chronopharmacodynamics and chronotoxicology) even after chronic application or constant infusion. Moreover, there is clear evidence that dose/concentration-response relationships can be significantly modified based on the time of day. Thus, circadian time has to be taken into account as an important variable influencing a drug's pharmacokinetics and its effects or side-effects and, as a result, drug doses need to be adjusted to meet the differing needs of target organs or tissues at various times of the day.

Recently, an orally administered drug for arthritis treatment has suggested a chronotherapeutic approach using a delay release system where the delay is scheduled to release the active ingredient at the beginning of an interleukin-6 (IL-6) cascade that is thought to cause early morning stiffness in rheumatoid arthritis patients. By attempting to synchronize the drug delivery with a biological cycle, it is believed that lower doses may be used to achieve desired results. However, this system does not overcome the limitations of delayed release systems described above.

Although it is possible to meet the requirements of chronopharmacology with pills, this requires an enormous amount of discipline by the patient to comply with the treatment regimen. As illustrated above, to achieve optimal results, many patients may need to wake up during the night to take their medication. Hence, there is a need for a reliable means of delivering bioactive agents in precisely timed and measured doses without inconveniencing the patient.

Currently, patient compliance (taking the proper dosages at the prescribed times) is a critical problem facing caregivers and pharmaceutical firms alike. Studies show that only about half of patients take medications at the times and in the dosages directed by their physician. It is reported that each year, 125,000 deaths and up to 20% of all hospital and nursing home admissions result from patient non-compliance. It is estimated that non-compliance results in additional healthcare costs in excess of $100 billion per year in the United States. These figures are even more pronounced for the elderly. Hence, a need exists for methodology and systems that increase patient compliance for the administration of a variety of compounds. This and other obstacles in the prior art are addressed by one or more embodiments of the present invention.

One successful chronotherapeutic approach involves synchronizing the administration of bioactive agents with the human body's natural circadian rhythms and addiction rhythms to counteract symptoms when they are likely to be at their worst by using an automated transdermal or other drug administration system. As described in U.S. Pat. No. 7,780,981, “Biosynchronous Transdermal Drug Delivery”, a device delivers varying dosages at various times, pursuant to a pre-programmed dosage profile. This ensures that peak drug concentrations are present in the bloodstream to offset peak disease and addiction symptoms arising from variances and fluctuation in the body's natural circadian rhythms.

While there have been tremendous advances in transdermal drug applications, the associated devices are limited to agents that retain biological activity following storage in the reservoir(s) linked with the device. To retain activity, the compound must, among other things, be compatible with the reservoir housing material, be stable in solution, be stable at variable temperatures, be stable for variable amounts of time and be protected against microbiological contamination. Meeting these, as well as other, requirements for use with a storage reservoir is challenging for some agents and completely prohibitive for others. Thus, a need exists for drug delivery methodology and systems that circumvent challenges associated with maintaining drug stability in a delivery device. This and other obstacles in the prior art are addressed by one or more embodiments of the present invention.

An additional hurdle for drug delivery systems that administer multiple doses of a bioactive agent is effectively ceasing delivery of the compound at a specified time. Delivery termination is typically accomplished by evaporation of the bioactive agent's carrier solution. However, this passive process is not instantaneous and can also be significantly variable. To aid in solvent evaporation, a desiccant can be employed. However, this does not remedy the drawbacks previously mentioned and, in addition, now the device must be equipped with sufficient housing for the desiccant and means to expose the solvent to the desiccant when drug delivery cessation is desired. Thus, a need exists for drug delivery methodology and systems that can actively control and quickly terminate delivery of the compound. This and other obstacles in the prior art are addressed by one or more embodiments of the present invention.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### SUMMARY OF THE INVENTION

Briefly stated, one embodiment of the present invention involves synchronizing the administration of compounds with the human body's natural biological rhythms to counteract symptoms of, including but not limited to, diseases, disorders and addictions when they are likely to be at their worst by using a bioactive agent administration system and device.

The present innovation is particularly useful for applications in which it is necessary and/or desirable to start the administration of a drug, stop the administration of a drug, and/or increase/decrease the dosage of a drug at a time when it is inconvenient or impossible for a patient to initiate the necessary actions. This is specifically advantageous for a wide variety of drug administration applications that benefit when administration is started, stopped, or changed while a person is sleeping.

On embodiment of the present invention includes a bioactive agent delivery device having a plurality of reservoirs wherein each reservoir houses a solvent. The device further includes a plurality of absorbent materials wherein each absorbent material is pretreated with a bioactive agent and a delivery mechanism operable to deliver to any of the plurality of absorbent materials a controlled portion of solvent from any of the plurality of reservoirs. Interposed between the plurality of absorbent materials and an epidermis of a human or animal operable is a diffusion layer operable to transfer the bioactive agent from the absorbent materials to the epidermis for delivery of the bioactive agent.

Additional features of the present invention include an embodiment wherein the solvent is a cessation solution operable to terminate delivery of the bioactive agent while in other versions of the invention the solvent is an initiation solution operable to initiate delivery of the bioactive agent. In yet other versions the solvent is a moderation solution operable to moderate a rate of delivery of the bioactive agent.

The solvent in one embodiment is also operable to facilitate transfer of the at least one bioactive agent from the at least one absorbent material pretreated with at least one bioactive agent to the diffusion layer. In one example the absorbent material is pretreated with a bioactive agent.

The invention disclosed herein can also include a timing mechanism communicatively coupled to the delivery mechanism that can generate the control signal delivering a solution from at least one of the plurality of reservoirs to at least one of the plurality of absorbent materials. This signal can be configured to follow an administration schedule customized for each patient. For example the programmed administration schedule can be synchronized with a biological rhythm or the like.

To aid in the delivery of the solvent each reservoir can include a pressure source such as compressed foam or a gas generation cell, or the like. The device of the present invention can deliver the bioactive agent through the epidermis using various means such as micro-needles, transdermal diffusion, iontophoresis, sonophoresis, electroporation, nanoporation, dermal abrasion, sub cutaneous delivery, heat, piezoelectric droplet jet dispensers, thermal droplet jet dispensers, and light and chemical permeation enhancers. Moreover the bioactive agent being administered can include such things as alprazolam, apomorphine, azelastine, buprenorphine, bupropion, clonidine, enalpril, estradiol, ethinyl estradiol, fentanyl, granisetron, insulin, lidocaine, memantine, methylphenidate, methamphetamine, nitroglycerine, nicotine, norethisterone acetate (NETA), norelgestromine, oxybutynin, pergolide, phenteramine, pramipexole, ramipril, ropinirole, rotigotine, scopolamine, selegiline, tecrine, testosterone, timolol and tolterodine.

In another embodiment of the present invention a bioactive agent delivery device includes a plurality of reservoirs that are fluidly coupled to the at least one of a plurality of absorbent materials. Each of these reservoirs can house a solution and a valve coupled each reservoir that can control delivery of the solution contained therein onto the absorbent material. Interposed between, and fluidly coupled to the at least one absorbent material, is a membrane that interfaces with the epidermis of a human. A delivery mechanism responsive to a first control signal operable delivers a first solution from a first of the plurality of reservoirs to the at least one absorbent material initiating delivery of a bioactive agent, and, responsive to a second control signal delivers a second solution from a second of the plurality of reservoirs to the at least one absorbent material. In one embodiment the second solution can modify the pH of the first solution terminating delivery of the bioactive agent.

The present invention, as described herein, further includes methodology for delivering a segmented bioactive agent. Such methodology includes storing in a reservoir one or more solvents and fluidly coupling to each reservoir an absorbent material such as blotter paper. One device can include multiple reservoirs and multiple absorbent materials. The method continues by segregating the absorbent materials and interposing a membrane between each absorbent material and the epidermis of a human. A delivery mechanism delivers a first controlled portion of one of the solvents from the reservoirs to an absorbent material establishing forming a bioactive agent. In one embodiment the absorbent material can be pretreated with a bioactive agent and the solution activates/hydrates the substance while in other embodiments the solvent contains the bioactive agent. With the presence of a bioactive agent established a dose is transferred from the absorbent material to the epidermis of the human via the membrane. Moreover a second release can occur from a second reservoir to a second absobant material that is segmented from the first. In such a way different bioactive agents can be administered or the same agent can be administer (subsequent administration) in a precisely controlled manner

The method of the present invention also includes delivery of a cessation solution operable to terminate delivery of the one or more bioactive agents. In once instance the solution modifies the pH of the bioactive agent to terminate delivery.

Embodiments of the present invention include, as will be described below a device and associated methodology to deliver a controlled dose of a bioactive agent to an individual and do so in alignment with temporal and bisynchronous requirements. These and other features of the present invention are described by way of example in the specification that follows.

### BRIEF DESCRIPTION OF THE DRAWINGS

The aforementioned and other features and objects of the present invention and the manner of attaining them will become more apparent, and the invention itself will be best understood, by reference to the following description of one or more embodiments taken in conjunction with the accompanying drawings, wherein:

FIG. 1 is a perspective view of an exemplary device useful for transdermal deliver of pharmaceutical agents according to one embodiment of the present invention;

FIG. 2 presents a top and side view of a transdermal drug delivery system using pre-treated blotter paper according to one embodiment of the present invention;

FIG. 3 shows a graph of suboptimal human plasma drug concentrations after multiple doses administered by a transdermal drug delivery device of the present invention;

FIG. 4 is a cross-sectional and partial bottom illustration of an exemplary device using a pressure means for implementing transdermal drug delivery according one embodiment of the present invention;

FIG. 5 is an exploded and side cutaway view of an exemplary device for implementing transdermal drug deliver according to one embodiment of the present invention;

FIG. 6 is a flowchart of one method embodiment for delivery of a bioactive agent using a transdermal drug delivery system of the present invention; and

FIG. 7 is a flowchart of another method embodiment for delivery of one or more bioactive agents using a transdermal drug delivery system of the present invention.

The Figures depict embodiments of the present invention for purposes of illustration only. One skilled in the art will readily recognize from the following discussion that alternative embodiments of the structures and methods illustrated herein may be employed without departing from the principles of the invention described herein.

### DESCRIPTION OF THE INVENTION

The reality of biological rhythms in animals, including humans, is well known. Biological rhythms are periodic fluctuations in biological characteristics over time, including: ultradian, which are cycles shorter than a day (for example, the milliseconds it takes for a neuron to fire, or a 90-minute sleep cycle); circadian, which are cycles lasting approximately 24 hours (for example, sleeping and waking patterns); infradian, which are cycles longer than 24 hours (for example, monthly menstruation); and seasonal cycles, such as seasonal affective disorder (SAD), which causes depression in susceptible people during the short days of winter.

Circadian (approximately 24-hour) rhythms include the production of biological molecules such as endorphins, gonadotropin releasing hormone (GnRH), cortisol and adrenaline, regulation of body temperature and heart rate, alteration of blood characteristics, such as stickiness, and behavioral changes such as wakefulness, sleep and periods of activity.

Research demonstrates that for particular conditions and diseases and the symptoms thereof, drug effects can be optimized by administration of specific (and often varying) dosages at defined times. This is known as chrono-pharmacology. Illustrating the importance of chrono-pharmacology, consider the following facts: asthma attacks are 100 times more likely to occur between 4:00 and 6:00 AM. Heart attacks and strokes are most likely to occur around 6:00 AM. Variant angina attacks occur 30 times more often in the middle of the night between 2:00 AM and 4:00 AM. Smokers experience the highest cravings immediately upon waking up. Lethargy and difficulty getting out of bed is highest immediately upon waking up early in the morning. Cold and flu symptoms peak during nighttime and early morning hours, when cold medications typically wear off.

Certain disease symptoms follow a daily pattern, peaking at specific times of day. It has been widely acknowledged that hormones, neurotransmitters and other intra-body compounds are released in varying amounts at different times of the day pursuant to daily patterns. It is believed that the failure of current transdermal systems to synchronize drug administration with the body's natural rhythms often lead to (i) severe side effects, including debilitating sleep disorders (in the context of nighttime nicotine administration, for example), (ii) ever increasing tolerance (in the case of nitroglycerin and other pharmaceuticals, for example), (iii) more expensive therapies, since more of a compound is needed when body rhythm-tailored dosing is not implemented. In addition, many addictions follow a daily pattern consistent with one's circadian rhythms. For example, studies have shown that, immediately upon waking, smoker's experience peak nicotine cravings. These intense cravings return after each meal, due to the interplay of serotonin release as a trained response to the culmination of a meal.

Innovations associated with the present invention facilitates precisely timed administration of bioactive agents so that peak drug levels are synchronized with times when symptoms are likely to be maximal or times when the compounds are most effective and/or better tolerated by the patient. The present invention is described in terms of a particular example drug delivery system that provides automated and precise control over dosing, with a multiple single-dose capability or the capacity to administer individual doses of one or more bioactive agents multiple times throughout a day or multi-day period.

Embodiments of the present invention are hereafter described in detail with reference to the accompanying Figures. Although the invention has been present disclosure has been made only by way of example and that those skilled in the art can resort to numerous changes in the combination and arrangement of parts without departing from the spirit and scope of the invention.

The terms and words used in the following description and claims are not limited to the bibliographical meanings, but, are merely used by the inventor to enable a clear and consistent understanding of the invention. Accordingly, it should be apparent to those of reasonable skill in the art that the following description of exemplary embodiments of the present invention are provided for illustration purpose only and not for the purpose of limiting the invention as defined by the appended claims and their equivalents.

It is to be understood that the singular forms “a,” “an,” and “the” include plural referents unless the context clearly dictates otherwise. Thus, for example, reference to “a bioactive agent” includes reference to one or more of such bioactive agents.

By the term “substantially” it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of a device in use or operation in addition to the orientation depicted in the figures. For example, if a device in the figures is inverted, elements described as “under” or “beneath” other elements or features would then be oriented over the other elements or features. Thus, the exemplary term “under” can encompass both an orientation of “over” and “under”. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In accordance with one embodiment of the present invention, substances with proven or suspected chrono-pharmacological efficiency are integrated into a miniaturized, automated, programmable device. This delivery system can be used for a variety of bioactive agents and is readily adapted to be worn on the forearm, ankle, or other convenient body location.

One particular implementation of the present innovation is consistent with commercial development of a miniaturized, automated and programmable noninvasive drug delivery device called the ChronoDose™ system, which is being developed by the assignee of the present invention. This system controls the amount of bioactive agents exposed to the skin in a precisely timed manner according to a programmed administration schedule that implements a desired dosage profile. Thus, one embodiment of the present innovation enables the precise control of both the dose amount and time of application, pursuant to an easily-set preprogrammed dosage profile as well as the ability to control the cessation or slowing (moderation) of treatment.

One aspect of bioactive agent administration systems is the compatibility of compounds with storage in the delivery device. To be compatible, an agent must remain stable (i.e. retain its physical, chemical and therapeutic properties) for the duration of storage and usage by the recipient. The innovation of the present invention described herein includes a means by which a bioactive agent can be stored in a delivery system without compromising the stability of the compound, yet be subsequently administered under precise control with the associated bio-synchronous system.

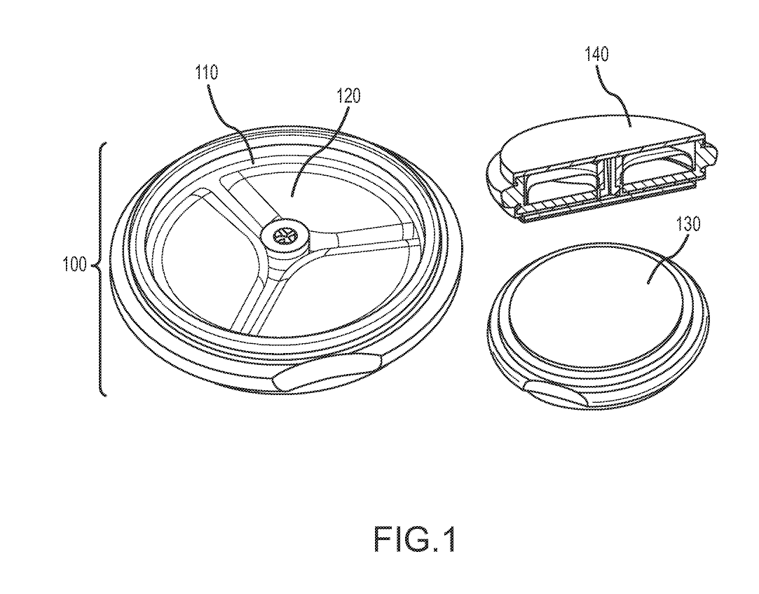

One embodiment useful for transdermal drug delivery, according to the present invention, comprises an absorbent material pretreated or combined with a bioactive agent. FIG. 1 presents a high-level prospective view of a transdermal drug delivery device according to the present invention in which a pretreated absorbent material can be combined with a bioactive agent. The transdermal drug delivery system 100 includes one or more reservoirs 110 housed by a supporting structure 120 that is encapsulated on the upper side by a pressure source 140 or means to initiate a solvent/agent interaction and on the lower side by a permeable membrane 130 that acts as an interface with an individual's dermis. In such a situation one of the reservoirs provides a solution of a drug or solvent. The solution is pumped or otherwise applied per a dose schedule to a blotter paper for diffusion and interaction with a skin interfacing membrane.

As used herein, the term “absorbent material” refers to a material having the capacity or tendency to absorb another substance. The term “bioactive agent” refers herein to a substance that has an effect on a living organism.

Bioactive agents (or combinations thereof) suitable for transdermal drug delivery include but are not limited to hormonal contraceptives, pain relievers, antidepressants, stimulants for treating, for example, ADHD, biopharmaceuticals, active agents useful in treating chronic conditions, opioids, alprazolam, apomorphine, azelastine, alprostadil, buprenorphine, bupropion, clonidine, dexamethasone, dextroamphetamine, diclofenac, dihydrotestosterone, enalpril, estradiol, androgen/estradiol, estrodiol/progestin, testosterone/estradiol, ethinyl estradiol, fentanyl, flurbiprofen, glucagon-like peptide 1, glucagon-like peptide 2, granisetron, insulin, lidocaine, memantine, methylphenidate, methamphetamine, nitroglycerine, nicotine, norethisterone acetate (NETA), norelgestromine, oxybutynin, parathyroid hormone, pergolide, phenteramine, pramipexole, ramipril, ropinirole, rotigotine, scopolamine, selegiline, tecrine, testosterone, timolol, tolterodine, tulobuterol, and vaccines.

Conditions effectively treated with bioactive agents delivered transdermally include, but are not limited to, addiction, anxiety, allergies, depression, hypertension, nausea, diabetes, neuralogic pain, Alzheimer's disease, obesity, smoking cessation, urinary incontinence, Parkinson's disease, motion sickness, male hypogonadism and female sexual dysfunction.

In one embodiment of the present innovation, an absorbent material pretreated with bioactive agent is a component of a drug delivery system that also comprises a reservoir having a solution and a membrane permeable to the bioactive agent. The absorbent material pretreated with bioactive agent is interposed between the reservoir and the membrane. In response to a control signal, the solution is dispensed from the reservoir and is received by the absorbent material pretreated with bioactive agent. The solution is operable to facilitate transfer of the bioactive agent from the absorbent material to the membrane. In accordance with one embodiment of the present invention, the membrane is in contact with the epidermis of a host (a human or an animal). As the membrane is permeable to the bioactive agent, this compound diffuses through the membrane from the absorbent material and into the epidermis of the host. In this way, bioactive agent is transdermally administered to the host of the drug delivery system.

According to one embodiment of the present innovation, a solution is actively dispensed from one or more reservoirs by a delivery mechanism that comprises a mode of force. In one embodiment of the present invention, the mode of force is pressure which, for example, includes but is not limited to compressed foam, one or more gas generating cells, and the like. By applying pressure to the solution in the reservoir, the solution is dispensed from the reservoir through a valve operable to control the flow of the solution from the reservoir to the absorbent material pretreated with bioactive agent. In accordance with another embodiment of the present innovation, a pump functions as the delivery mechanism operable to dispense the solution from the reservoir to the absorbent material pretreated with bioactive agent.

Another feature of the present invention includes a timing mechanism that is communicatively coupled to the delivery mechanism and operable to generate a control signal to dispense the solution from the reservoir to the absorbent material pretreated with bioactive agent at a predetermined time. In one embodiment of the present innovation, the timing mechanism communicatively coupled to the delivery mechanism is configured to generate the control signal according to a programmed administration schedule. In another embodiment of the present invention the control signal to administer the drug could be transmitted to the device that can augment or control internal functionality. As an example, a drug delivery device of the present invention can be coupled to a human and programmed to administer a compound during sleep. By using the system of the present invention, the patient is appropriately dosed without having to be inconvenienced by waking up and personally administering the bioactive agent.

One contemplated consumer product is a device that addresses sleep and awakening disorders. Most people experience difficulty and discomfort when waking early in the morning. According to a 2002 National Sleep Foundation poll, 49% of US adults age 18-29 and 41% of US adults age 30-64 have trouble awaking in the morning. There are 165,000,000 adults in the US alone age 18-64, meaning approximately 74,250,000 US adults age 18-64 have trouble awaking in the morning.

One implementation of the present invention allows individuals, while asleep, to have an over-the-counter (OTC) or prescription stimulant automatically administered during a 1-2 hour pre-wakeup period. In one embodiment of the present innovation, an absorbent material pretreated with a stimulant is used as the bioactive agent source for the drug delivery system. With the use of this device, the stimulant dose is timed such that the concentration in the host will reach peak levels immediately prior to waking up. Thus, upon waking, the individual is alert and feels well-rested. Consequently, one embodiment of the present invention eliminates the typical discomfort or difficulty associated with getting up early. This functionality is attractive to, for example, employees getting up early for work to ensure punctuality as well as anyone wanting to offset morning tiredness associated with, for example, a late night, a long night, illness, or jet lag.

Another exemplary application contemplated by the present innovation is smoking cessation. To support smokers in their efforts to quit, nicotine replacement has been the most frequently used therapy. Smokers report that the craving for a cigarette is greatest immediately upon waking. In fact, the time between awakening and indulging in a cigarette is the best indicator of addiction. For most smokers, this time period is only a few minutes.

Current nicotine patches cause severe sleep disturbances because they are designed to release this compound steadily throughout the night to ensure sufficient morning nicotine levels to offset the strong morning craving. This is often detrimental to the nighttime use of these patches. Often, users remove their nicotine patch in the evening before they go to bed. This eliminates sleep disturbances but results in residual nicotine levels that are insufficient to offset the strong morning craving. Accordingly, many users relapse when they arise. Thus, current nicotine patch therapy presents the user with a difficult decision, having to choose either sleep disturbances or limited relief from strong morning nicotine cravings.

An exemplary product, accordingly to one embodiment of the present innovation, is the Nicotine ChronoDose™ system. In accordance to one embodiment of the present invention, an absorbent material pretreated with nicotine is utilized as the bioactive agent source in the drug delivery device. The system administers nicotine (or nicotine analogs or any other smoking cessation compound including but not limited to Zyban) automatically during the period immediately prior to waking This relieves the smoker's peak early morning craving without causing sleep disturbances.

In another embodiment of the present innovation, the programmed administration schedule is synchronized with at least one biological rhythm of a human or an animal. By way of example, an application to treat cold or flu is detailed.

Cold and flu symptoms are worst in the middle of the night and early morning because the concentration of the hormone cortisol, a key inflammation fighter, is lowest during that time. Current nighttime cold or flu medications lose efficacy by early morning, and thus, people suffering from a cold or flu are often unpleasantly awoken by an increase in symptom severity, cutting sleep short.

In one embodiment, according to the present invention, an absorbent material pretreated with cold or flu medication is employed as the bioactive agent source for the drug delivery system. The device, programmed and put on before bedtime, automatically delivers a dose of medication in the early morning hours to more effectively combat the peak cold or flu symptoms that occur in the morning. Accordingly, users experience less severe cold or flu symptoms during the morning hours, do not have their sleep cycle cut short and wake up feeling symptom-free. One embodiment of the present invention administers prescription or OTC cold and/or flu medication alone. In accordance with another embodiment of the present innovation, the prescription or OTC cold and/or flu medication is delivered in combination with vitamins and immune system boosters to provide a comprehensive treatment for cold and flu ailments.

An exemplary embodiment of the present invention comprises a drug administration device operable to deliver multiple doses of a bioactive agent or doses of multiple bioactive agents. According to this system, the device includes multiple individual bioactive agent sources. Each source comprises absorbent material pretreated with a bioactive agent that interacts with a solvent. In one embodiment of the present innovation, each individual source contains the same amount of bioactive agent. In another embodiment of the present invention, the individual sources contain varying amounts of the same bioactive agent. Yet another embodiment, in accordance with the present innovation, includes individual sources that comprise varying bioactive agents. When a dose of bioactive agent is required as determined by a treatment protocol, one or more reservoirs contained within the delivery device dispense a solution operable to facilitate transfer of the bioactive agent from an individual absorbent material to the epidermis of the host. When an additional dose of bioactive agent is required, one or more reservoirs contained within the delivery device dispense a solution operable to facilitate an additional transfer of the bioactive agent from another individual absorbent material to the epidermis of the host. In this way, multiple doses are delivered with a single drug delivery device. The time period between doses can vary, for example, from minutes to days.

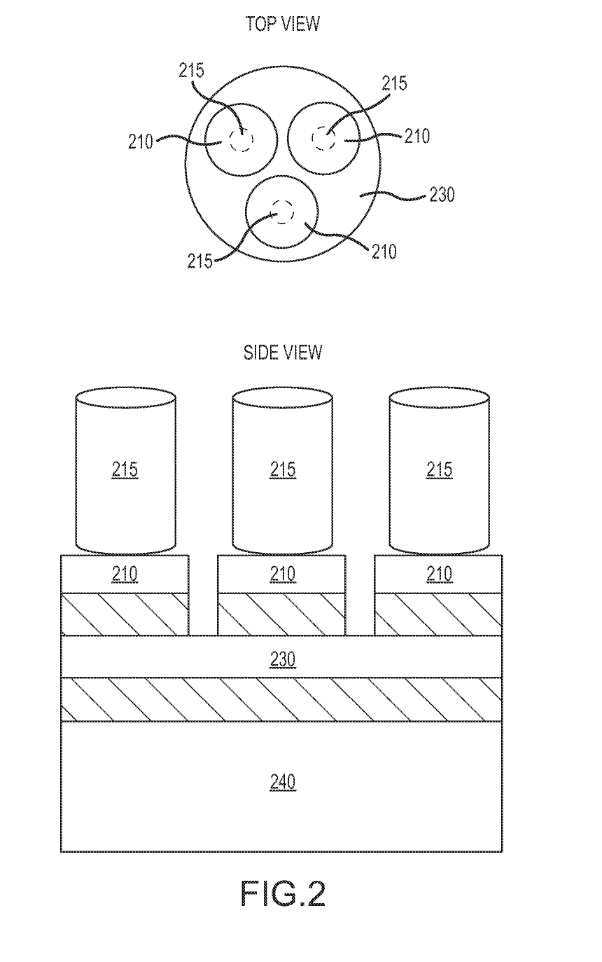

One embodiment of the present invention includes a circular device, illustrated in FIG. 2 configured to house and deliver multiple doses of a bioactive agent. The “Top View”, the transdermal drug delivery system of FIG. 2 illustrates the relative orientation of, according to one embodiment of the present innovation, three individual sites in which each site is operable to deliver a dose of bioactive agent, over an exemplary circular-shaped device design. The “Side View” illustrates one embodiment of the various components of each individual site and the relationship of these sites to additional components of the drug delivery system operable to facilitate transfer of bioactive agent from the individual pretreated sites to the epidermis of the host. Each individual site comprises an absorbent material 210 pretreated with bioactive agent. This drug source 210 has a first surface 212 and a second surface 216, wherein the first bioactive agent source surface 212 is fluidly coupled to a reservoir 215 containing a solution. This solution, once dispensed from the reservoir, is operable to facilitate transfer of the bioactive agent from the individual bioactive agent source 210 to a site-specific membrane 220 via a second surface 216. This site-specific membrane 220 is permeable to the bioactive agent and has a first surface 221 and a second surface 228. The first site-specific membrane surface 221 is in fluidic contact with the second bioactive agent source surface 216. The second site-specific membrane surface 228 is in contact with a first surface 232 of an untreated absorbent material 230 operable to facilitate transfer of the bioactive agent from the site-specific membrane 220 to a membrane 235 in contact with the epidermis 240 of the host. This membrane 235 has a first surface 232 and a second surface 234. The untreated absorbent material 230 is interposed between the second site-specific membrane surface 220 and the first surface 225 of the membrane 235. As can be seen with reference to FIG. 2, the second membrane surface 234 is in contact with the epidermis 240 of the host.

In accordance with one embodiment of the present innovation and as illustrated in FIG. 2, when a dose of bioactive agent is required, a solution is dispensed from a reservoir 215 to an individual absorbent material 210 pretreated with bioactive agent. The solution is operable to facilitate transfer of the bioactive agent from an individual absorbent material 210 to a site-specific membrane 220. The bioactive agent diffuses from the site-specific membrane 220 to an untreated absorbent material 230 (blotter paper) operable to facilitate transfer of the bioactive agent from the site-specific membrane 220 to a membrane 235 in contact with the epidermis 240 of the host. In this way, the bioactive agent is delivered from the bioactive agent delivery device to the host through the skin (transdermal drug delivery). When an additional dose is required, the method described above is repeated with another solution, and individual absorbent material pretreated with bioactive agent. According to one embodiment of the present invention shown in FIG. 2, the system is capable of administering three doses to a host. In each case the bioactive agent present in the pretreated absorbent material 210 may be the same or they may be distinct.

When a bioactive agent is administered transdermally, the dose received by the host is directly dependent upon the surface area of the drug delivery system component that is in contact with the epidermis of the host. In one embodiment of the present invention detailed in FIG. 2, the surface area of the membrane 235 that is in contact with the skin 240 differs in size from the surface area of the absorbent material 210 pretreated with bioactive agent. This configuration allows for the surface area of the drug delivery device component that is in contact with the epidermis of the host to be modified independently from the surface area of the absorbent material pretreated with bioactive agent. This aspect, in turn, permits dose adjustments by simply modifying the treated surface area of the system that is in contact with the host.

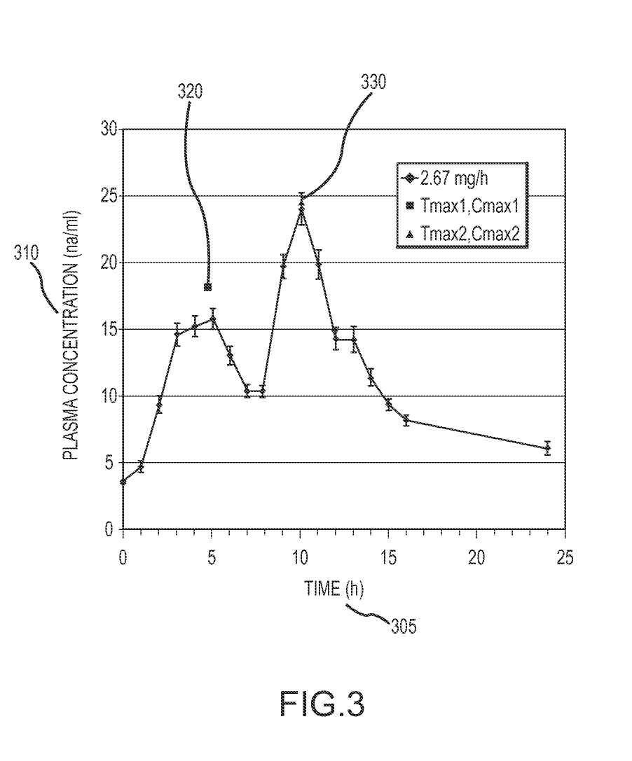

A caveat of current bioactive agent delivery systems comprising absorbent material, is that a residual amount of the drug remains within the absorbent material following each dose. During a subsequent dosing process, the residual drug is delivered in addition to the new dose, increasing the concentration of active drug in that dose. FIG. 3 illustrates an exemplary drug administration profile of one implementation of the present invention. The graph of FIG. 3 shows plasma levels 340 of drug after two doses in nanograms per milliliter on the vertical axis 310 and time on the horizontal axis 305. The time period on the graph spans the application of two dosing events. The dose of the applied drug for each event, that is the amount of solvent released to the pre-treated absorbent material, and the degree upon which the pretreated absorbent material is impregnated, are identical. As can be seen in FIG. 2, two plasma peaks are indicated. A first peak 320 associated with the 1^{st} dose and a 2^{nd} peak 330 associated with the second dosing. The residual drug remaining within the absorbent material from the 1^{st} dose combines with the 2^{nd} dose, creating a higher peak (Cₘₐₓ2) 330 of active drug. According to one embodiment of the present innovation, the doses of bioactive agent are individualized and, as a result, this system solves the problem detailed above by facilitating dose compensation to account for residual drug in the absorbent material to ensure that the desired drug concentration is administered. That is to say, the administration of the second dose is adjusted to account for the residual effect of the prior dose(s) so as to achieve predictable and desirable plasma levels.

One exemplary and contemplated application of the present invention is assistance with smoking cessation. A nicotine replacement system according to the present invention is, in one example, worn for several days at a time. In accordance to one embodiment of the present innovation, multiple individualized absorbent materials pretreated with nicotine (or similar nicotine cessation treatment drug) are used as the bioactive agent source within the drug delivery device. The system is programmed to release the prescribed medication in a daily rhythmic pattern to offset peaks in a smoker's cravings. This implementation reduces nicotine dependency by administering preprogrammed levels of medication (in one embodiment, nicotine) pursuant to typical smoking patterns. For instance, many smokers report that cravings for a cigarette are greatest upon waking up, after lunch, mid afternoon, after dinner and before bedtime. This implementation of the present invention will automatically release larger doses of medication to offset peak cravings and no medication when cravings are typically at a minimum. The present innovation delivers medication in a preprogrammed manner for each treatment regimen and aligned with an individual's biometric patterns. In this way, drug administration is independent of user involvement other than the initial application and initiation of the device, thus decreasing inconvenience to the user and increasing dosing compliance and, therefore, agent efficacy.

This exemplary implementation of the present invention is a tremendous advancement in nicotine replacement therapy and is far superior to the old-technology systems that simply release a constant amount of nicotine continuously throughout the day and night. According to one embodiment of the present innovation, an individual can systematically decrease nicotine tolerance without increasing dependence (the result of a constant flow approach) so as to better wean a smoker off nicotine completely. The present invention enables a smoker to better ‘tailor-down’ and decrease the amount of nicotine he/she needs to quit.

Modern smoking cessation treatment often includes much more than simple nicotine replacement therapy. Programs also comprise weight control, diet and psychological support. One embodiment of the present invention fits ideally into these programs, as this innovation addresses the key component of being able to quit smoking by efficiently countering nicotine withdrawal symptoms while eliminating the negative side effects of current nicotine replacement therapy systems, namely sleep disturbances.

Another application for one embodiment of the present invention includes weight control and vitamin and herbal supplementation. According to one embodiment of the present innovation, multiple individualized absorbent materials pretreated with weight loss vitamins and supplements are used as the bioactive agent source within the drug delivery system. Subsequently, these compounds are administered in small distinct doses many times over a predetermined period of time. As the body in small dosages absorbs vitamins and supplements, once-a-day products are not maximally effective because excess dosages are excreted unused. Implementation of the present invention precisely controls the timing and dosage of small but distinct amounts of agents, in this case vitamins and supplements, during a 24-hour period to ensure that vitamins and supplements are constantly bioavailable for optimal absorption and cellular function. Greater doses are automatically released prior to mealtimes to counter appetite cravings, resulting in a much more effective diet program.

In yet another embodiment, the present invention is applicable for the treatment of cold or flu symptoms by using multiple individualized absorbent materials pretreated with cold or flu medications as the bioactive agent source within the drug delivery device. The system releases agents every, for example but not limited to, 2 hours throughout the night, with a higher dosage of compounds being released in the morning to combat established middle-of-the-night and early morning symptoms, which are often the most severe.

The present innovation, according to yet another embodiment, can also employed for angina treatment. Research shows that variant angina occurs 30 times more often between 2:00 a.m. and 4:00 a.m. (‘critical angina phase’) than at any other time of the day. Nitroglycerin effectively combats angina attacks, if timely administered in optimal doses. Current nitroglycerin patches exist, but they can only release a constant amount of nitroglycerine steadily over time. Current patches cannot tailor the release of nitroglycerine to optimize treatment by releasing more nitroglycerine precisely during the critical angina phase to offset these peak symptoms.

In addition, nitroglycerine loses its effectiveness and requires higher and higher dosages when administered constantly. Like many forms of medication, our bodies become tolerant. Current systems cannot stop or decrease the release of nitroglycerine when disease symptoms are lowest. Thus, these current ‘dumb’ patches cannot offset the critical angina phase by releasing more of the drug when optimally needed, nor can it cease nitroglycerine administration when the host does not need this compound. Current systems address a ‘one-dose-fits-all’ type of scenario once each “dumb” patch is applied to the patient.

The methodology and system in accordance with the present invention utilize multiple individualized absorbent materials pretreated, in this example, with nitroglycerine as the bioactive agent source within the drug delivery device. The automated transdermal system administers more nitroglycerine during the critical angina phase to ensure adequate offset of these symptoms and less nitroglycerine when it is not needed so that tolerance does not build up. Thus, one embodiment of the present innovation utilizes a “smart” medication system to offset peak critical phases in the disease cycle arising from the human body's circadian rhythm.

In one embodiment of the present invention, the programmable automated transdermal drug delivery system is worn around the wrist (or the forearm arm or ankle) much like a watch. Continuing with the present example, the system releases nitroglycerine in optimal dosages at times that are optimally synchronized with the patient, pursuant to a preprogrammed and tailored dosage profile.

The nitroglycerin system in accordance with the present innovation has three primary advantages over current nitroglycerin patches. First, the system utilizes its core competitive advantage to automatically and precisely release nitroglycerin in peak amounts to offset the peak symptoms of morning attacks occurring during the critical angina phase. Current nitroglycerine patches have release rates that stay constant and do not increase to offset critical phases, and do not decrease as symptoms decrease. Second, our system solves the tolerance issue by releasing less (or no) nitroglycerin in off-peak hours, and then releasing nitroglycerin at just the right time so that it is present during critical periods, without increasing tolerance. Third, our system accomplishes 1 and 2 above automatically, without the need for a patient to wake up to take a drug at this critical phase, which does away with the need for any increased patient compliance.

As a result, the nitroglycerin system of the present invention represents an ideal delivery system for patients who use nitroglycerin regularly for the treatment and/or the prevention of heart attacks and strokes. Patient compliance regarding the timing and dose of heart attack medication is crucial. Patient non-compliance with physician's instructions for this is often a cause of re-hospitalization, according to the US Department of Health and Human Services. The system solves this problem, and will decrease the need for re-hospitalization by dramatically increasing patient compliance.

This system can be either a ‘wear-each-night-and-remove-in-the-morning’ system, whereby it only releases nitroglycerine automatically to offset the critical angina phase in the morning, or a ‘total solution’ system, that is worn for a period of 24 hours to several days and administers nitroglycerine in tailored amounts at precise times synchronized with the body's circadian rhythm.

Yet another application for one embodiment of the present invention is asthma therapy. According to one embodiment of the present innovation, multiple individualized absorbent materials pretreated with albuterol, tolobuterol, salmeterol, beta 2 agonist or any other antiarrhythmic drug are used as the bioactive agent source within the drug delivery device. An automated transdermal delivery system automatically administers a morning dose of compound to combat the peak symptoms of morning asthma attacks known as the ‘morning dip’.

Asthma attacks occur 100 (one hundred) times more often between the hours 4 A.M. and 6 A.M., when most people are asleep. This is due to the early morning deterioration of respiratory function known as the ‘morning dip’, which is the time of day that respiratory function is at its lowest. These early morning asthma attacks cause great distress to sufferers and care providers. One embodiment of the present innovation effectively combats the morning dip by releasing more medication at this time to offset these peak morning symptoms. In other words, the optimized and synchronized drug delivery system of the present invention varies the level of drug in the bloodstream so that drug concentrations are highest when respiratory function is at its lowest.

The asthma drug delivery system, according to one embodiment of the present invention, has two primary advantages over current patches. First, the system of the present invention utilizes its core competitive advantage to automatically and precisely release albuterol or other asthma drugs in peak amounts to offset the peak symptoms associated with the morning dip. Current patches have release rates that stay constant and do not increase to offset this peak critical phases, and do not decrease as symptoms decrease. Second, various embodiments of the present invention accomplishes the release and establishment of a peak plasma level automatically, without the need for a patient to wake up to take a drug at this critical phase, increasing patient compliance. Thus, the advantages offered by the implementation of one embodiment of our present innovation are extremely beneficial to patients with moderate to severe asthma.

In yet another application, the present invention is utilized for the treatment of hypertension. Blood pressure also varies throughout the day. For example, blood pressure surges upon waking and is lower by 20 to 30 percent while sleeping. In accordance with one embodiment of the present innovation, multiple individualized absorbent materials pretreated with clondine or another hypertension drug are used as the bioactive agent source within the drug delivery device. The automated transdermal drug delivery system is programmed to release, for example but not limited to, clondine in the morning to combat the peak symptoms of A.M. hypertension.

Current clonidine patches release the drug continuously over time. Variable dosing is currently not an option. Therefore, having the advantage of administering more medication when a patient needs it most can mean the difference between life and death, especially in patients with moderate to severe high blood pressure.

The automated transdermal system for hypertension, according to one embodiment of the present innovation, has several advantages over current patches. For example, the present invention automatically and precisely releases clondine or other hypertension drugs so as to present a peak amounts in the blood stream to offset the peak symptoms associated with the dangerous morning hypertension symptoms. Current hypertension patches have release rates that stay constant and do not increase to offset this peak critical phases, and do not decrease as symptoms decrease. And as with other implementations, this entire process can take place without the need for a patient to wake up to take a drug at this critical phase, enhancing compliance.

Yet another application for the present invention is treatment for depression, Alzheimer's disease and Attention Deficit Disorder. Multiple individualized absorbent materials pretreated with selegiline or like medication, an effective monoamine oxidase (MAO) inhibitor, can be used as the bioactive agent source within the drug delivery device of the present invention. The automated transdermal drug delivery system of the present invention provides an automated morning release of a drug such as selegiline to combat the peak symptoms of morning depression without the side effect of sleep disturbances. The system in accordance with the present invention is initiated before bed with a predetermined wake time and releases the drug an hour or two before waking, so symptoms of morning depression are corrected without subjecting the patient to sleep disturbances.

Primary negative side effects of current selegiline patches are abnormal dreams, insomnia, and difficulty sleeping. By specifically refraining from administering selegiline during the restful portion of a night's sleep, and utilizing our system's core competitive advantage to administer drug roughly an hour before waking, the system of the present invention eliminates this negative side effect and can effectively offset the critical phase of morning symptoms of depression.

In one embodiment of the present invention, the absorbent material pretreated with the bioactive agent is prepared by saturating the material in a drug/solvent formulation for an amount of time specific to ensure the desired amount of drug is present within the absorbent material. This saturation may be accomplished by methods including but not limited to soaking and spraying. Following saturation, the solvent is evaporated. An exemplary process used for this purpose includes but is not limited to utilization of a cold nitrogen evaporator. The evaporation of the solvent results in an absorbent material pretreated with bioactive agent that can be stored or further processed for use with the drug delivery system of the present invention.

Advantages of utilizing absorbent material pretreated with a bioactive agent versus a reservoir containing a fluidic bioactive agent formulation in a drug delivery device include but are not limited to, 1) absorbent material pretreated with a bioactive agent is less susceptible to degradation, as it is maintained separately from other formulation components such as water, 2) solution(s) stored in the reservoir and operable to facilitate transfer of the bioactive agent from a pretreated absorbent material may be less expensive than a solution required to maintain the integrity of a formulation containing the active agent, and 3) handling of absorbent material pretreated with bioactive agent during the drug delivery device manufacturing and assembly process can be less hazardous and more convenient than handling active agent-containing reservoirs.

In one embodiment of the present invention, the absorbent material is blotter paper, while in another embodiment of the present invention, the absorbent material pretreated with bioactive agent is heat/pressure laminated using web unwind/rewind and or pick-and-place technology followed by web flow through a heat/pressure laminator.

In one embodiment of the present innovation, transdermal diffusion of the bioactive agent is terminated by evaporation of the bioactive agent's carrier solution. To aid in solvent evaporation, a desiccant is employed, according to one embodiment of the present invention while in yet another embodiment of the present innovation, the drug delivery cartridge comprises a venting system to facilitate solvent evaporation.

While evaporation of the solvent containing the bioactive agent is an effective means to cease or diminish drug delivery, this passive process is not instantaneous and can also be significantly variable. To improve drug delivery cessation control, one embodiment of the present invention includes a solution within one of the plurality of reservoirs operable to terminate or, in another embodiment, diminish, transdermal diffusion of the bioactive agent.

One embodiment of the present invention significantly improves drug delivery cessation by modulating the pH of a solution in contact with the bioactive agent. This strategy is based on the observation that a drug is rendered charged or uncharged relative to its pKₐ value. Since only the uncharged form of a drug can permeate across lipophilic membranes including the epidermis, drug diffusion through the skin can be terminated by altering the pH of the solution in contact with the bioactive agent.

In accordance with one embodiment of the present invention, at least one of a plurality of reservoirs stores a cessation solution with a pH operable to terminate transdermal diffusion of the bioactive agent. In an exemplary device comprised of three reservoirs (1, 2 and 3), reservoirs 1 and 2 include a bioactive agent formulation and reservoir 3 includes a formulation operable to alter pH. The bioactive agent formulation included in reservoir 1 is dispensed from the reservoir to absorbent material fluidly coupled to the reservoir at a specified time when the first drug administration is desired. Subsequently, as the solution infiltrates the absorbent material the first dose of bioactive agent is delivered to the host by transdermal diffusion. When an additional dose of bioactive agent is required, the bioactive agent formulation included in reservoir 2 is dispensed from the reservoir to absorbent material fluidly coupled to the reservoir at a specified time when the second drug administration is desired. Consequently, the second dose of bioactive agent is delivered to the host by transdermal diffusion.

In this example, after the second dose of bioactive agent has been exposed to the epidermis of the host for an allotted period of time, cessation of the transdermal diffusion of the compound is desired. Accordingly, the formulation included in reservoir 3 is dispensed from the reservoir to absorbent material fluidly coupled to the reservoir at a specified time when termination of drug administration is required. The formulation in reservoir 3 is operable to alter the pH of the solution in contact with the skin such that diffusion of the bioactive agent through the skin is halted. This example details a drug delivery device comprised of 3 reservoirs, however, as is clear to a person skilled in the relevant art, a plurality of any number of reservoirs is within the scope of the present innovation.

In accordance with one embodiment of the present invention, at least one of a plurality of reservoirs stores a cessation solution with a pH operable to terminate transdermal diffusion of the bioactive agent and at least one of the plurality of reservoirs stores an initiation solution with a pH operable to initiate transdermal diffusion of the bioactive agent. In an exemplary device comprised of three reservoirs (1, 2 and 3), reservoir 1 includes a bioactive agent formulation, reservoir 2 includes a formulation with a pH operable to terminate transdermal diffusion of the bioactive agent and reservoir 3 includes a formulation with a pH operable to initiate or enhance transdermal diffusion of the bioactive agent following delivery cessation. The bioactive agent formulation included in reservoir 1 is dispensed from the reservoir to absorbent material fluidly coupled to the reservoir at a specified time when the first drug administration is desired. Subsequently, the first dose of the bioactive agent is delivered to the host by transdermal diffusion. After the first dose of the bioactive agent has been exposed to the epidermis of the host for an allotted period of time, cessation of the transdermal diffusion of the compound is desired. Accordingly, the formulation included in reservoir 2 is dispensed from the reservoir to absorbent material fluidly coupled to the reservoir at a specified time when termination of drug administration is required.

The formulation in reservoir 2 is, in one embodiment, operable to alter the pH of the solution in contact with the skin such that diffusion of the bioactive agent through the skin is halted. In accordance with one embodiment of the present innovation, the formulation included in reservoir 2 is operable to make the pH of the solution in contact with the skin more acidic. When an additional dose of bioactive agent is required, the formulation included in reservoir 3 is dispensed from the reservoir to absorbent material fluidly coupled to the reservoir at a specified time when the second drug administration is desired. In accordance with one embodiment of the present invention, the formulation included in reservoir 3 is operable to make the pH of the solution in contact with the skin more basic in order to counteract the acidic formulation dispensed from reservoir 2. In this way, transdermal drug administration is re-initiated following transdermal delivery cessation. This example details a drug delivery device comprised of 3 reservoirs, however, as is clear to a person skilled in the relevant art, a plurality of any number of reservoirs is within the scope of the present innovation.

According to yet another embodiment of the present invention, at least one of a plurality of reservoirs stores a cessation solution with a pH operable to terminate or diminish transdermal diffusion of the bioactive agent. In an exemplary device comprised of three reservoirs (1, 2 and 3), reservoirs 1 and 2 include a solution operable to facilitate diffusion of a bioactive agent from an absorbent material pretreated with bioactive agent to the epidermis of a host and reservoir 3 includes a formulation operable to alter pH. The solution included in reservoir 1 is dispensed from the reservoir to absorbent material pretreated with bioactive agent and fluidly coupled to the reservoir at a specified time when the first drug administration is desired. Subsequently, the first dose of bioactive agent is delivered to the host by transdermal diffusion. When an additional dose of bioactive agent is required, the solution included in reservoir 2 is dispensed from the reservoir to absorbent material pretreated with bioactive agent and fluidly coupled to the reservoir at a specified time when the second drug administration is desired. Consequently, the second dose of bioactive agent is delivered to the host by transdermal diffusion. After the second dose of bioactive agent has been exposed to the epidermis of the host for an allotted period of time, cessation of the transdermal diffusion of the compound is desired. Accordingly, the formulation included in reservoir 3 is dispensed from the reservoir to untreated absorbent material fluidly coupled to the reservoir at a specified time when termination of drug administration is required. The formulation in reservoir 3 is, in one embodiment, operable to alter the pH of the solution in contact with the skin such that diffusion of the bioactive agent through the skin is halted. This example details a drug delivery device comprised of 3 reservoirs, however, as is clear to a person skilled in the relevant art, a plurality of any number of reservoirs is within the scope of the present innovation.

In yet another embodiment, according to the present invention, at least one of a plurality of reservoirs stores a cessation solution with a pH operable to terminate transdermal diffusion of the bioactive agent and at least one of the plurality of reservoirs stores an initiation solution with a pH operable to initiate transdermal diffusion of the bioactive agent. In an exemplary device comprised of three reservoirs (1, 2 and 3), reservoir 1 includes a solution operable to facilitate diffusion of a bioactive agent from an absorbent material pretreated with bioactive agent to the epidermis of a host, reservoir 2 includes a formulation with a pH operable to terminate transdermal diffusion of the bioactive agent and reservoir 3 includes a formulation with a pH operable to initiate transdermal diffusion of the bioactive agent following delivery cessation. The solution included in reservoir 1 is dispensed from the reservoir to absorbent material pretreated with bioactive agent and fluidly coupled to the reservoir at a specified time when the first drug administration is desired. Subsequently, the first dose of bioactive agent is delivered to the host by transdermal diffusion. After the first dose of bioactive agent has been exposed to the epidermis of the host for an allotted period of time, cessation of the transdermal diffusion of the compound is desired. Accordingly, the formulation included in reservoir 2 is dispensed from the reservoir to untreated absorbent material fluidly coupled to the reservoir at a specified time when termination of drug administration is required. The formulation in reservoir 2 is, in one embodiment, operable to alter the pH of the solution in contact with the skin such that diffusion of the bioactive agent through the skin is halted. In accordance with one embodiment of the present innovation, the formulation included in reservoir 2 is operable to make the pH of the solution in contact with the skin more acidic. When an additional dose of bioactive agent is required, the formulation included in reservoir 3 is dispensed from the reservoir to absorbent material pretreated with bioactive agent and fluidly coupled to the reservoir at a specified time when the second drug administration is desired. In accordance with one embodiment of the present invention, the formulation included in reservoir 3 is operable to make the pH of the solution in contact with the skin more basic in order to counteract the acidic formulation dispensed from reservoir 2. In this way, transdermal drug administration is initiated following transdermal delivery cessation. This example details a drug delivery device comprised of 3 reservoirs however, as is clear to a person skilled in the relevant art, a plurality of any number of reservoirs is within the scope of the present innovation.

Another embodiment, according to the present invention, comprises absorbent material pretreated with a pH modulator. When a solution is dispensed from the reservoir and contacts the pH modulator within the pretreated absorbent material, the pH of the solution is altered wherein the solution becomes operable to terminate or diminish transdermal diffusion of the bioactive agent. In this way, the pH of the solution is operable as an “off switch”, terminating transdermal diffusion of the bioactive agent, or “dimmer switch” to diminish the transdermal diffusion of the bioactive agent.

When a solution is dispensed from another reservoir and contacts the pH modulator within the pretreated absorbent material, the pH of the solution is altered again, wherein the solution becomes operable to initiate or increase transdermal diffusion of the bioactive agent. In this way, the pH of the solution is operable as an “on switch”, or the brightening of a “dimmer switch” initiating additional transdermal diffusion of the bioactive agent.

Another embodiment of the transdermal drug delivery device of the present invention is an interface for coupling to a human or animal that includes at least one absorbent material, at least one reservoir housing bounding a reservoir space storing a solution, wherein the reservoir housing includes at least one through hole with a valve operable to control delivery of the solution from the reservoir space to the at least one absorbent material, at least one delivery mechanism operable to deliver the at least one solution from the at least one reservoir space to at least one absorbent material in response to a control signal, and at least one membrane interposed between the at least one absorbent material and the human or animal, operable to deliver the bioactive agent from the at least one absorbent material to the human or animal.

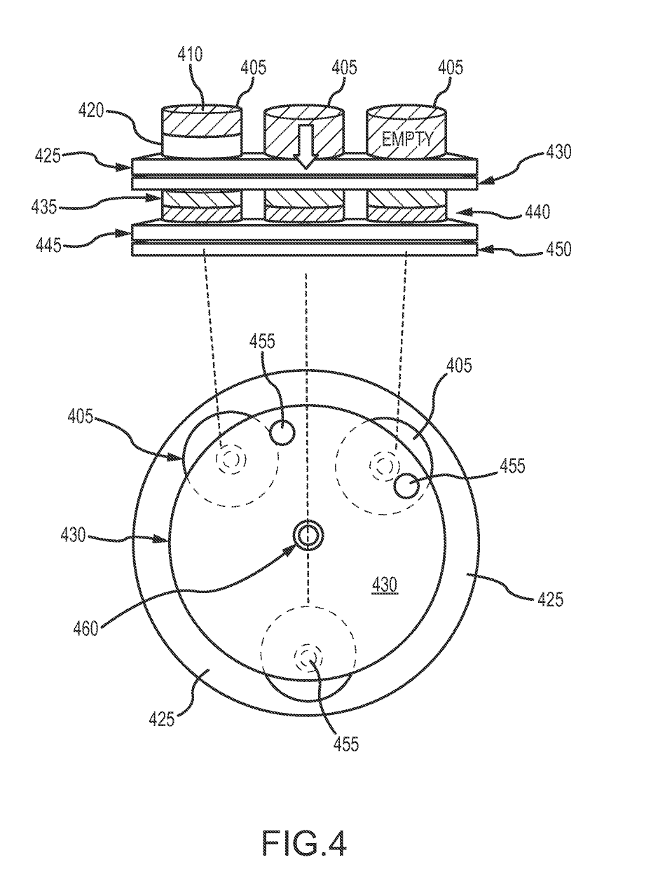

One exemplary embodiment of this version of the invention is shown in FIG. 4. FIG. 4 presents a side and corresponding partial bottom view of a bio-synchronous drug delivery device of the present invention. This embodiment includes multiple reservoirs 405 arranged in a circular manner each housing a fluidic solution 420. The depiction shown in FIG. 4 includes three (3) reservoirs 405. On top of each reservoir 420 is a pressure source 410 such as compressed foam, an array of chemical gas generating pistons or the like. When actuated, the pressure source 410 presses on an individual reservoir, dispensing its contents 420 to one or more fluidly coupled absorbent materials 435 in contact with one or more membranes 440 operable to deliver the bioactive agent to the epidermis of a host. As shown the rightmost reservoir 405 is empty with the pressure source 410 expended. The center reservoir 405 indicates and ongoing process of the pressure source 410 expending the solution 420 to the underlying absorbent material 435.

Continuing with additional reference to the partial bottom view of FIG. 4, one of reasonable skill in the relevant art can see that a plurality of reservoirs 405 (in this example there are three) are symmetrically coupled to a reservoir mounting disk 425. In this example the mounting disk would be permeable. Beneath each reservoir 405 is an absorbent material 435 suitable for collecting and dispersing the medication or solvent contained within the reservoir 405. Interposed between the absorbent material 435, the combination reservoir 405, and mounting disk 425 is a rotary disk 430. In the embodiment shown in FIG. 4, the rotary disk 430 and mounting 425 are rotatable coupled via a drive shaft 460 centrically positioned. The rotary disk 430 includes an access port 455 for each reservoir. In this case and as can be seen in FIG. 4, the rotary disk 430 includes three (3) access ports. These access ports 455 are asymmetrically positioned on the rotary disk 430 such that only one access port will align with any one reservoir 405 at any one time. In the example shown in FIG. 4, the lowest most reservoir 405 (the center reservoir as seen in the side view) is aligned with its access port 455, while the rotary disk 430 blocks access to the other reservoirs 405. One skilled in the art will recognize that other configurations of the access ports can be established so as to provide access to none, multiple, or all of the reservoirs. In one embodiment of the present innovation, a motor operable to rotate the rotary disk 430 in relation to the mounting disk 425 rotates the rotary disk 430. In another embodiment of the present innovation, the rotary disk 430 is rotated by spring tension operable to rotate the rotary disk 430 in relation to the mounting member 425.

Each reservoir 405 includes a pressure source such as a foam pad 410 that exerts pressure on the medication/solvent 420 within the reservoir 405. Upon the rotary disk 430 aligning a port 455 with a reservoir 405 under pressure, the contents 420 can be dispensed through the port 455. For example, each port 455 can possess a rubber or similar seal that provides access to the solvent 420 when aligned but otherwise seals the reservoir 405. Beneath the rotary disk 430 is a diffusion layer 445 (blotter paper) and an membrane 450 that interfaces with the dermis of the patient. According to one embodiment of the present invention the membrane interfacing with the epidermis of a user can be comprised of ethylene vinyl acetate (eva) including substantially 2% to 40% vinyl acetate. Interposed between the diffusion layer 445, according to one embodiment, exists pretreated or a medication storage layer 435 followed by another eva membrane 440. In such an implementation the reservoir can include a solvent that, when released interacts with a bioactive agent.

In one embodiment of the present invention, absorbent material pretreated with bioactive agent is in contact with untreated absorbent material. An unwind/rewind system is employed to pretreat the absorbent material, align the pretreated absorbent material with the untreated absorbent material (and intervening membrane layers as needed), and then heat/pressure laminate the materials together. In yet another embodiment of the present innovation, absorbent material pretreated with bioactive agent is picked-and-placed onto a moving web of untreated absorbent material and laminated.

In one embodiment of the present innovation, the rotary disk 430 is in contact with absorbent material 435, the absorbent material 435 is in contact with a membrane 440 and the membrane 440 is in contact with the epidermis of the host.

The rotary disk 430 can also be rotated in relation to the mounting disk 425 by a motor. Alternatively, for this purpose, the rotary disk is rotated in relation to the mounting disk by utilizing spring tension. A solenoid (under microcontroller control) can lift a pawl at a designated time, allowing the disk to rotate (powered by the compressed spring or motor), supporting the pawl on the end of the disk until an alignment notch aligns with the pawl. The pawl then falls into the notch, stopping the rotation of the rotary disk and resulting in the alignment of a rotary disk valve with a reservoir value. This alignment is operable to dispense the solution from the reservoir.

According to one embodiment of the present invention, clock like movement is utilized to control the rotation of the rotary disk. A separate ring on the periphery of the device is rotated, advancing the clock movement. When started, activated by a mechanical release (go switch), the clock movement unwinds and rotates the rotary disk, resulting in the alignment of a rotary disk valve with a reservoir value. This alignment is operable to dispense the solution from the reservoir. The separate ring to set a start time is patterned after the time ring on a submariner watch. The ring on the watch has a series of numbers printed on it. It is rotated in relation to the watch face, allowing one to time 10 minutes, 20 minutes as the minute hand moves. The concept for the present innovation is similar for the ring itself, but differs in that the ring would pre-set a watch movement internal to the device to a time of day, resulting in rotary disk alignment with a reservoir at a specified time of day.

According to one embodiment of the present invention, transdermal compound penetration is assisted using one or more skin permeation technologies from the group comprising: micro-fabricated structures commonly referred to as micro-needles, sub cutaneous delivery, iontophoresis, sonophoresis, dermal abrasion, electroporation, nanoporation, piezoelectric droplet jet dispenser, thermal droplet jet dispenser, heat, light and chemical permeation enhancers and/or a wide range of nanostructures and substances known as nanotechnology or any combination of these techniques. Chemical permeation enhancers include but are not limited to oleic acid, amino acids, oleyl alcohol, long chain fatty acids, propylene glycol, polyethylene glycol, isopropanol, ethoxydiglycol, sodium xylene sulfonate, ethanol, N-methylpyrrolidone, laurocapram, alkanecarboxylic acids, dimethylsulfoxide, polar lipids, N-methyl-2-pyrrolidone, and the like, which increase the permeability of the skin to the active material and permit the active material to penetrate through the skin and into the bloodstream. Pharmaceutically acceptable compositions may be combined with one or more agents including, but not limited to, alcohols, moisturizers, humectants, oils, emulsifiers, thickeners, thinners, surface active agents, fragrances, preservatives, antioxidants, vitamins, or minerals. Pharmaceutically acceptable compositions may also be combined with a polymeric substance including, but not limited to, ethylcellulose, hydroxypropyl cellulose, ethylene/vinylacetate, polyvinyl pyrrolidone, and the like, to provide the composition in gel form, which may be dissolved in solvent such as methylene chloride, evaporated to the desired viscosity, and then applied to a membrane.

The membrane can be any of the conventional materials including but not limited to silicones and siloxanes, polyethylene, polypropylene, ethylene-vinyl acetate (EVA) copolymer, polyurethane and the like. In accordance with one embodiment of the present innovation, the membrane is comprised of ethylene-vinyl acetate (EVA) including 19% vinyl acetate.

In yet another embodiment of the present invention, various novel formulation technologies such as microspheres, nanoparticles, hydrogels, liposomes and nano-emulsions are incorporated to achieve enhanced trans-dermal drug delivery. Specific examples of agents that may be employed for this purpose both alone and in combination include but are not limited to, adrenergics (adrenaline and similar drugs), adrenocortical steroids (dexamethasone acetate and similar drugs), alcohol deterrents (disulfuram and similar drugs), anabolic steroids (nandrolone cycloate and similar drugs), analgesics (oxycodone hydrochloride and similar drugs), anesthetics, in adjunct to sodium oxybate (ketamine hydrochloride and similar drugs), antagonists (naltrexone and similar drugs), anterior pituitary suppressants (danazol and similar drugs), anti-acne medication (adapalene and similar drugs), anti-allergics (astemizole and similar drugs), anti-anginals (amolopidine besylate and similar drugs), anti-anxietics (ondasetron hydrochloride and similar drugs), anti-arthritics (lodelaben and similar drugs), anti-asmatics (montelucast sodium and similar drugs), anti-bacterials (amifioxacin mesylate and similar drugs), anticholinergics (glycopyrrolate and similar drugs), anti-convulsants (phenytoin and similar drugs), anti-diabetics (buformin and similar drugs), anti-emetics (alosetron hydrochloride and similar drugs), anti-epileptics (felbamate and similar drugs), anti-estrogens (tamoxifen citrate and similar drugs), anti-fungals (miconazole and similar drugs), anti-histaminics (clemastin fumerate and similar drugs), anti-infectives (acyclovir and similar drugs), anti-inflammatories (tolmetin sodium and similar drugs) and anti-neoplastics (ambomycin and similar drugs).

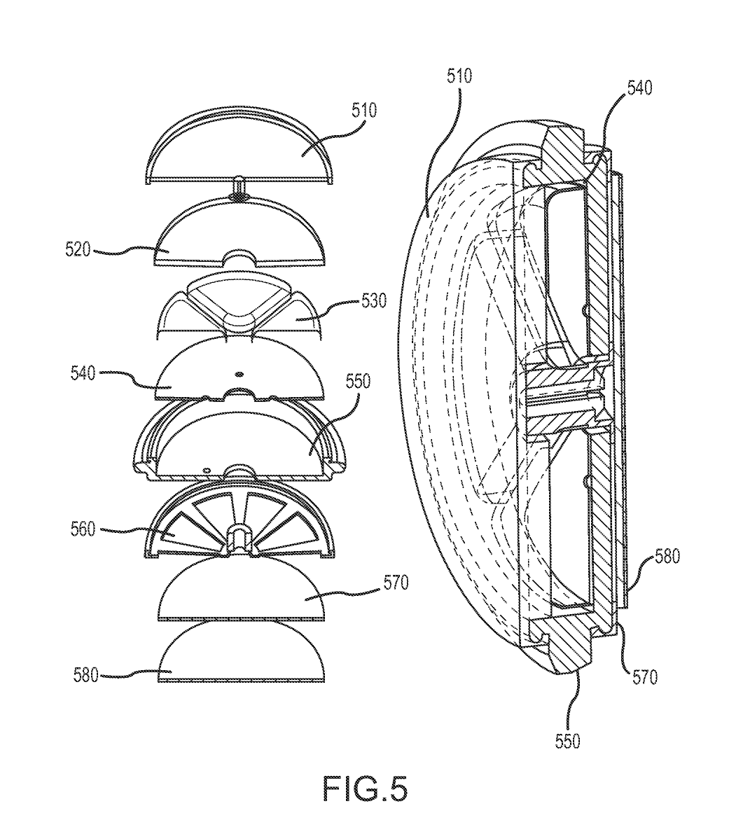

FIG. 5 presents an exploded and cutaway view of another embodiment of the present invention. The transdermal drug delivery system includes a top cap 510 followed by a foam layer 520 that can be used as a pressure source. As shown in FIG. 4 the foam layer or pressure source can be compartmentalized into individual foam segments associated with each reservoir. Beneath the foam layer 520 is a reservoir 530 suitable for holding a fluidic solvent/solution. A reservoir-mounting disk 540 is interposed between the reservoir 530 and the rotary disk 550. Under the rotary disk 550 is a diffusion support structure 560 and a diffusion layer 570 or blotter paper. Finally, the diffusion layer 570 is coupled to an eva membrane 580 or the like. In another embodiment, a pretreated paper layer can be coupled to or laminated to the diffusion layer 570.

The transdermal drug delivery systems of the present invention can be utilized to treat a wide variety of ailments. The systems described above introduce a means by which one of a plurality of solvent reservoirs can be independently and partially released such that the solvent contained within can either be dispersed onto the dermis of a patient or interact with a pre-treated bioactive paper. One aspect of the invention described herein, therefore, is the methodology by which to provide variable yet controlled application of transdermally applied medication.

Included in the description that follows are flowcharts depicting examples of the methodology that may be used to chronologically and/or bio-synchronously deliver transdermal medication to a patient. In the following description, it will be understood that each block of the flowchart illustrations, and combinations of blocks in the flowchart illustrations, can be implemented by computer program instructions. These computer program instructions may be loaded onto a computer or other programmable apparatus to produce a machine such that the instructions that are executed on the computer or other programmable apparatus create means for implementing the functions specified in the flowchart block or blocks. These computer program instructions may also be stored in a computer-readable memory that can direct a computer or other programmable apparatus to function in a particular manner such that the instructions stored in the computer-readable memory produce an article of manufacture, including, instruction means that implement the function specified in the flowchart block or blocks. The computer program instructions may also be loaded onto a computer or other programmable apparatus to cause a series of operational steps to be performed in the computer or on the other programmable apparatus to produce a computer implemented process, such that the instructions that execute on the computer or other programmable apparatus provide steps for implementing the functions specified in the flowchart block or blocks.

Accordingly, blocks of the flowchart illustrations support combinations of means for performing the specified functions and combinations of steps for performing the specified functions. It will also be understood that each block of the flowchart illustrations, and combinations of blocks in the flowchart illustrations, can be implemented by special purpose hardware-based computer systems that perform the specified functions or steps, or combinations of special purpose hardware and computer instructions.

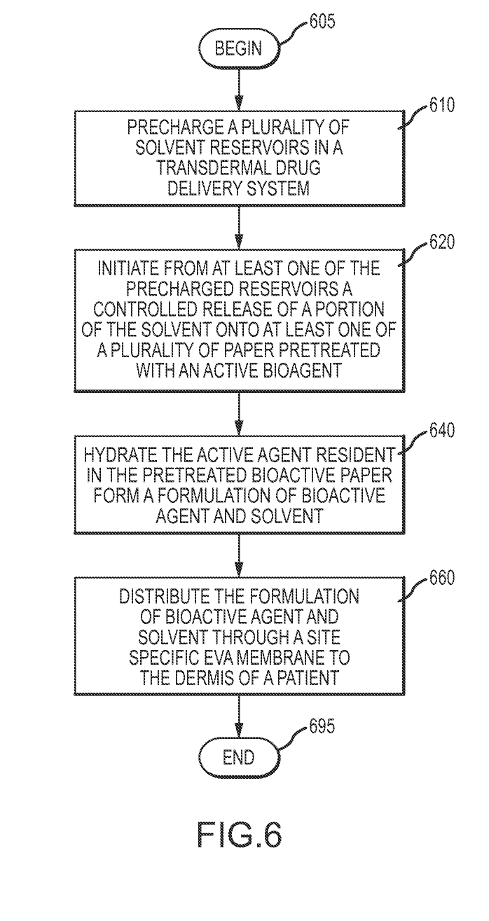

FIG. 6 presents a flowchart of one method embodiment for transdermally delivering a bioactive agent to a patient, according to the present invention. The process begins 605 with pre-charging 610 a plurality of solvent reservoirs in a transdermal drug delivery system. As previously described one embodiment of the present invention includes a plurality of solvent reservoirs positioned so that each reservoir is associated with a piece of blotter paper (or the like) pretreated with a bioactive agent. The method for providing transdermal delivery of medication continues by initiating 620 a controlled release of at least a portion of the solvent housed within one of the plurality of reservoirs onto one of the pretreated bioactive pieces of blotter paper.

The release of the solvent onto the pretreated paper hydrates 640 and activates the bioactive agent present on the paper. One of reasonable skill in the relevant art will recognize that the composition of the solvent and the pretreated bioactive paper can complement each other and be of various compositions.

As the bioactive agent on the paper is activated the formulation or combination of solvent and bioactive agent are distributed 660 through one or more membranes and diffusion papers so as to arrive in contact with the dermis of a patient. Once in contact with the patient, transdermal delivery of the bioactive action can begin, ending the process 695.

Consistent with the teachings herein, the solvent contained within each reservoir can be released in a controlled manner. By doing so, a piece of blotter paper pretreated with a bioactive agent can be re-hydrated or reactivated by reinitiating a solvent release. Since the blotter paper has already been activated a residual of bioactive agent may remain making a subsequent solvent release produce a high concentration of the bioactive agent. The present invention thus can, through a single reservoir, initiate multiple solvent releases to modulate medication concentration.

Another feature of the present invention is to isolate several pretreated bioactive pieces of blotter paper with separate solvent reservoirs on a single system. By doing so, the initiation of a solvent release of individual reservoirs along with its associated bioactive blotter paper can be provide the ability to deliver different formulations to the patient. Indeed the intermediate layer of eva membrane on top of a diffusion layer of one embodiment of the present invention allows a rehydrated (reactivated) drug to flow in a larger diffusion later for delivery to the patient without flowing laterally into neighboring pre-treated sites. Thus each reservoir provides a segregated dose and formulation.

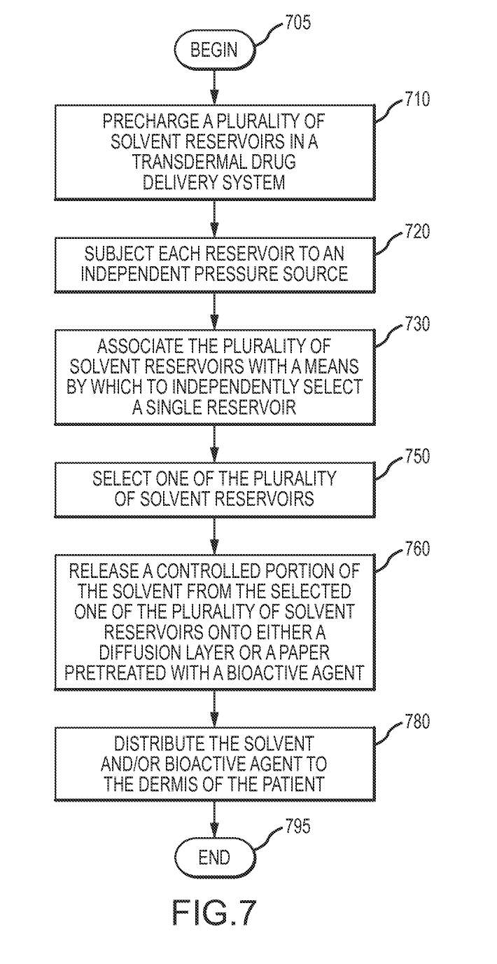

FIG. 7 provides a flowchart of yet another methodology for delivering transdermal medication to a patient according to one embodiment of the present invention. The process begins 705 again with establishing a plurality of pre-charged 710 reservoirs in a transdermal drug delivery system. Each of the reservoirs can possess the same solvent or they may possess different solvents that can be released in a controlled manner to interact with each other. For example, and as described above, one reservoir may contain a bioactive agent while another a pH modulator that can slow or cease transdermal diffusion of the bioactive agent. Thus the present invention increases the versatility and applicability of the transdermal drug delivery system.

The methodology described in FIG. 7 continues by associating 720 each reservoir with an independent pressure system. Rather than rely on capillary or gradient diffusion, one embodiment of the present invention initiates active delivery of the solvent to a blotter paper for diffusion onto the patient's dermis. The pressure charged reservoirs of the present invention are further associated 730 with a means by which the delivery of the solvent can be independently controlled. In one embodiment of the present invention reservoirs can house different substances so as to control/modulate transdermal delivery of a selected medication. Once means of doing this is through pH adjustment of the bioactive agent formulation. Thus, after the application of a bioactive agent onto the dermis of a patient, diffusion can be slowed or ceased by modulating its pH. This can be accomplished using a controller release of a secondary solvent housed in a different reservoir.

Turing back to FIG. 7 the process or delivering a bioactive agent to the dermis of a patient continues by selecting 750 one of the plurality of solvent reservoirs and releasing 760 a controlled portion of the solvent. This controlled release typically flows to either a diffusion layer for immediate interaction with the dermis of a patient or onto an intermediate layer of pretreated paper. This paper can be pretreated with a bioactive agent as described in connection with the methodology of FIG. 6.

Ultimately the formulation is distributed 780 to the dermal layer of the patient where the medication (formulation) is absorbed ending 795 the process. The methodologies presented herein are exemplary in nature and should not be viewed as limiting any other possible methods by which to deliver bioactive agent for transdermal absorption consistent with this invention. Indeed the techniques described herein can be combined and/or isolated to provide differing means by which to provide optimized care.

While the invention has been particularly shown and described with reference to embodiments, it will be understood by those skilled in the art that various other changes in the form and details may be made without departing from the spirit and scope of the invention.

While there have been described above the principles of the present invention in conjunction with examples, it is to be clearly understood that the foregoing description is made only by way of example and not as a limitation to the scope of the invention. Particularly, it is recognized that the teachings of the foregoing disclosure will suggest other modifications to those persons skilled in the relevant art. Such modifications may involve other features that are already known per se and which may be used instead of or in addition to features already described herein. It should be understood that the scope of the disclosure herein also includes any novel feature or any novel combination of features disclosed either explicitly or implicitly or any generalization or modification thereof which would be apparent to persons skilled in the relevant art, whether or not such relates to the same invention as claimed in any claim and whether or not it mitigates any or all of the same technical problems as confronted by the present invention. The Applicant hereby reserves the right to formulate claims to such features and/or combinations of such features during the prosecution of the present application or of any further application derived therefrom.

We claim:

1. A bioactive agent delivery device, comprising:
a plurality of reservoirs wherein each reservoir houses a solvent;
a plurality of absorbent materials wherein each absorbent material is pretreated with a bioactive agent;
a delivery mechanism operable to deliver to any of the plurality of absorbent materials a controlled portion of solvent from any of the plurality of reservoirs; and
a diffusion layer interposed between the plurality of absorbent materials and an epidermis of a human or animal operable to transfer the bioactive agent from the absorbent materials to the epidermis for delivery of the bioactive agent.

2. The bioactive agent delivery device according to claim 1, wherein the solvent is a cessation solution operable to terminate delivery of the bioactive agent.

3. The bioactive agent delivery device according to claim 1, wherein the solvent is an initiation solution operable to initiate delivery of the bioactive agent.

4. The bioactive agent delivery device according to claim 1, wherein the solvent is a moderation solution operable to moderate a rate of delivery of the bioactive agent.

5. The bioactive agent delivery device according to claim 1, wherein the solvent is operable to facilitate transfer of the at least one bioactive agent from the at least one absorbent material pretreated with at least one bioactive agent to the diffusion layer.

6. The bioactive agent delivery device according to claim 1, wherein the plurality of absorbent material is pretreated blotter paper.

7. The bioactive agent delivery device according to claim 1, wherein the diffusion layer is fluidly coupled to the plurality of absorbent materials.

8. The bioactive agent delivery device according to claim 1, wherein the diffusion layer comprises untreated blotter paper and membrane.

9. The bioactive agent delivery device according to claim 1, further comprising a membrane interposed between the plurality of absorbent materials and the diffusion layer.

10. The bioactive agent delivery device according to claim 1, further comprising a timing mechanism communicatively coupled to the delivery mechanism and operable to generate the control signal to deliver the solution from at least one of the plurality of reservoirs to at least one of the plurality of absorbent materials.

11. The bioactive agent delivery device according to claim 10, wherein the timing mechanism communicatively coupled to the delivery mechanism is configured to generate the control signal according to a programmed administration schedule.

12. The bioactive agent delivery device according to claim 11, wherein the programmed administration schedule is synchronized with at least one biological rhythm of the human or the animal.

13. The bioactive agent delivery device according to claim 1, wherein the at least one reservoir comprises a pressure source and the delivery mechanism comprises a valve configured to control release of portion of the solvent from the reservoir.

14. The bioactive agent delivery device according to claim 13, wherein the pressure source comprises compressed foam.

15. The bioactive agent delivery device according to claim 13, wherein the pressure source comprises a gas generation cell.

16. The bioactive agent delivery device according to claim 1, wherein the diffusion layer includes a membrane comprised of ethylene vinyl acetate (eva) including substantially 2% to 40% vinyl acetate.

17. The bioactive agent delivery device according to claim 1, wherein each of the plurality of reservoirs is operable to administer a plurality of doses of solvent.

18. The bioactive agent delivery device according to claim 1, wherein delivery of the bioactive agent through the epidermis is assisted using one or more from the group consisting of micro-needles, transdermal diffusion, iontophoresis, sonophoresis, electroporation, nanoporation, dermal abrasion, sub cutaneous delivery, heat, piezoelectric droplet jet dispensers, thermal droplet jet dispensers, and light and chemical permeation enhancers.

19. The bioactive agent delivery device according to claim 1, wherein the at least one bioactive agent is chosen from the group consisting of alprazolam, apomorphine, azelastine, buprenorphine, bupropion, clonidine, enalpril, estradiol, ethinyl estradiol, fentanyl, granisetron, insulin, lidocaine, memantine, methylphenidate, methamphetamine, nitroglycerine, nicotine, norethisterone acetate (NETA), norelgestromine, oxybutynin, pergolide, phenteramine, pramipexole, ramipril, ropinirole, rotigotine, scopolamine, selegiline, tecrine, testosterone, timolol and tolterodine.

20. The bioactive agent delivery device according to claim 1, wherein the solvent in at least one of the reservoirs is a cessation solution having a hydroalcoholic formulation comprising approximately 0.1 to 30% lactic acid.

21. The bioactive agent delivery device according to claim 1, wherein the delivery mechanism is a rotary disk having a plurality of unidirectional fluidic ports.

22. The bioactive agent delivery device according to claim 1, wherein the rotary disk is operable to align at least one of the unidirectional fluidic ports with at least one of the reservoirs and at least one of the absorbent materials and wherein responsive to the unidirectional fluid port, the at least one reservoir and at least one absorbent material being aligned, solvent from the at least one reservoir transfer to the at least one absorbent material and activates the bioactive agent.

23. A bioactive agent delivery device comprising:
at least one absorbent material;
a plurality of reservoirs fluidly coupled to the at least one absorbent material, where each reservoir stores a solution and wherein each reservoir includes a valve operable to control delivery of the solution contained therein to the at least one absorbent material;
at least one membrane interposed between and fluidly coupled to the at least one absorbent material and an epidermis of a human or an animal; and
at least one delivery mechanism responsive to a first control signal operable to deliver a first solution from a first of the plurality of reservoirs to the at least one absorbent material initiating delivery of a bioactive agent, and, responsive to a second control signal operable to deliver a second solution from a second of the plurality of reservoirs to the at least one absorbent material modifying the pH of the first solution and terminating delivery of the bioactive agent.

24. The bioactive agent delivery device of claim 23, wherein the at least one absorbent material is blotter paper.

25. The bioactive agent delivery device of claim 23, wherein the first solution includes the bioactive agent.

26. The bioactive agent delivery device of claim 23, wherein the at least one absorbent material is pretreated with the bioactive agent.

27. The bioactive agent delivery device of claim 23, wherein at least one of the plurality of reservoirs includes a solution containing the bioactive agent.

28. The bioactive agent delivery device of claim 23, wherein at least one of the plurality of reservoirs includes a solution void of the bioactive agent.

29. The bioactive agent delivery device of claim 23, wherein evaporation operates to cease delivery of the bioactive agent.

30. The bioactive agent delivery device of claim 29, wherein evaporation is assisted by a desiccant operable to facilitate evaporation.

31. The bioactive agent delivery device of claim 29, wherein evaporation of is assisted by a vent operable to facilitate evaporation.

32. The bioactive agent delivery device of claim 23, wherein at least one of the plurality of reservoirs houses a cessation solution operable to terminate delivery of the bioactive agent.

33. The bioactive agent delivery device of claim 32, wherein the pH of the cessation solution is operable to terminate delivery of the bioactive agent of the bioactive agent.

34. The bioactive agent delivery device of claim 23, wherein at least one of the plurality of reservoirs stores an initiation solution operable to initiate delivery of the bioactive agent of the bioactive agent.

35. The bioactive agent delivery device of claim 23, wherein the pH of the first solution is operable to initiate delivery of the bioactive agent.

36. The bioactive agent delivery device of claim 23, wherein at least one of the plurality of reservoirs comprises a pressurized reservoir and the delivery mechanism comprises a valve configured to control release of the solution from the pressurized reservoir.

37. The bioactive agent delivery device of claim 23, wherein the delivery mechanism comprises a pressure source.

38. The bioactive agent delivery device of claim 23, wherein the delivery mechanism comprises a pump.

39. The bioactive agent delivery device of claim 23, wherein the delivery mechanism is mechanically driven.

40. The bioactive agent delivery device of claim 23, wherein the at least one membrane is comprised of ethylene vinyl acetate (EVA) including substantially 2% to 40% vinyl acetate.

41. The bioactive agent delivery device of claim 23, wherein the first solution is the bioactive agent.

42. The bioactive agent delivery device of claim 41, wherein delivery of the bioactive agent through the skin is assisted using one or more from the group consisting of micro-needles, iontophoresis, sub cutaneous delivery, transdermal diffusion, sonophoresis, electroporation, nanoporation, dermal abrasion, heat, piezoelectric droplet jet dispensers, thermal droplet jet dispensers, and light and chemical permeation enhancers.

43. The bioactive agent delivery device of claim 41, wherein the bioactive agent is chosen from the group consisting of alprazolam, apomorphine, azelastine, buprenorphine, bupropion, clonidine, enalpril, estradiol, ethinyl estradiol, fentanyl, granisetron, insulin, lidocaine, memantine, methylphenidate, methamphetamine, nitroglycerine, nicotine, norethisterone acetate (NETA), norelgestromine, oxybutynin, pergolide, phenteramine, pramipexole, ramipril, ropinirole, rotigotine, scopolamine, selegiline, tecrine, testosterone, timolol and tolterodine.

44. A method for delivery of one or more bioactive agents, the method comprising:
storing in a plurality reservoirs one or more solvents;
fluidly coupling to each of the plurality of reservoirs one of a plurality of absorbent materials;
segregating the plurality of absorbent materials;
interposing at least one membrane between each of the plurality of absorbent materials and an epidermis of a human or an animal;
delivering by a delivery mechanism a first controlled portion of the one or more solvents from the plurality of reservoirs to a first absorbent material establishing a first bioactive agent in the first absorbent material;
transferring by the at least one membrane a first dose of the first bioactive agent from the first bioactive agent pretreated absorbent material to the epidermis of the human or the animal;
delivering by the delivery mechanism a second controlled portion of the one or more solvents from the plurality of reservoirs to a second absorbent material establishing a second bioactive agent in the second absorbent material; and
transferring by the at least one membrane a second dose of the second bioactive agent from the second bioactive agent pretreated absorbent material to the epidermis of the human or the animal.

45. A method for delivery of one or more bioactive agents of claim 44, wherein the first dose of bioactive agent and the second dose of bioactive agent are substantially identical bioactive agents.

46. A method for delivery of one or more bioactive agents of claim 44, wherein the first dose of bioactive agent and the second dose of bioactive agent are different bioactive agents.

47. A method for delivery of one or more bioactive agents of claim 44, wherein the first solution includes the first bioactive agent and wherein the second solution includes the second bioactive agent.

48. A method for delivery of one or more bioactive agents of claim 44, wherein the first absorbent material is pretreated with the first bioactive agent.

49. A method for delivery of one or more bioactive agents of claim 44, wherein the second absorbent material is pretreated with the second bioactive agent.

50. A method for delivery of one or more bioactive agents of claim 44, wherein at least one of the plurality of absorbent materials coupled to at least one of the plurality of reservoirs is pretreated with the bioactive agent

51. A method for delivery of one or more bioactive agents of claim 44, wherein at least one of the plurality of reservoirs includes a solution containing the bioactive agent.

52. A method for delivery of one or more bioactive agents of claim 44, wherein at least one of the plurality of reservoirs includes a solution void of the bioactive agent.

53. A method for delivery of one or more bioactive agents of claim 44, wherein evaporation operates to cease delivery of the bioactive agent.

54. A method for delivery of one or more bioactive agents of claim 53, wherein evaporation is assisted by a desiccant operable to facilitate evaporation.

55. A method for delivery of one or more bioactive agents of claim 53, wherein evaporation of is assisted by a vent operable to facilitate evaporation.

56. A method for delivery of one or more bioactive agents of claim 44, wherein at least one of the plurality of reservoirs houses a cessation solution operable to terminate delivery of the one or more bioactive agents.

57. A method for delivery of one or more bioactive agents of claim 56, wherein the pH of the cessation solution is operable to terminate delivery of the one or more bioactive agents.

58. A method for delivery of one or more bioactive agents of claim 44, wherein at least one of the plurality of reservoirs stores an initiation solution operable to initiate delivery of the one or more bioactive agents.

59. A method for delivery of one or more bioactive agents of claim 44, wherein the pH of the first solution is operable to initiate delivery of the bioactive agent.

60. A method for delivery of one or more bioactive agents of claim 44, wherein at least one of the plurality of reservoirs comprises a pressurized reservoir and the delivery mechanism comprises a valve configured to control release of the solution from the pressurized reservoir.

61. A method for delivery of one or more bioactive agents of claim 44, wherein the delivery mechanism comprises a pressure source.

62. A method for delivery of one or more bioactive agents of claim 44, wherein the delivery mechanism comprises a pump.

63. A method for delivery of one or more bioactive agents of claim 44, wherein the delivery mechanism is mechanically driven.

64. A method for delivery of one or more bioactive agents of claim 44, wherein the at least one membrane is comprised of ethylene vinyl acetate (EVA) including substantially 2% to 40% vinyl acetate.

65. A method for delivery of one or more bioactive agents of claim 44, wherein delivery of the one or more bioactive agents through the skin is assisted using one or more from the group consisting of micro-needles, iontophoresis, sub cutaneous delivery, transdermal diffusion, sonophoresis, electroporation, nanoporation, dermal abrasion, heat, piezoelectric droplet jet dispensers, thermal droplet jet dispensers, and light and chemical permeation enhancers.

66. A method for delivery of one or more bioactive agents of claim 44, wherein the one or more bioactive agents is(are) chosen from the group consisting of alprazolam, apomorphine, azelastine, buprenorphine, bupropion, clonidine, enalpril, estradiol, ethinyl estradiol, fentanyl, granisetron, insulin, lidocaine, memantine, methylphenidate, methamphetamine, nitroglycerine, nicotine, norethisterone acetate (NETA), norelgestromine, oxybutynin, pergolide, phenteramine, pramipexole, ramipril, ropinirole, rotigotine, scopolamine, selegiline, tecrine, testosterone, timolol and tolterodine.
